(19) United States
(12) Patent Application Publication
(10) Pub. No.: US 2022/0305268 A1
(43) Pub. Date: Sep. 29, 2022

(54) Neuromodulation Device

(71) Applicants: Galvani Bioelectronics Limited, Brentford, Middlesex (GB); Case Western Reserve University, Cleveland, OH (US)

(72) Inventors: Sarah Faridah Hassan, Brentford (GB); Stephen J. Lewis, Cleveland, OH (US); Sheridan McMurray, Brentford (GB); Arun Sridhar, Brentford (GB); Ibrahim Salman, Cleveland (US)

(21) Appl. No.: 17/619,567

(22) PCT Filed: Jun. 17, 2020
(86) PCT No.: PCT/GB2020/051455

§ 371 Date: Dec. 15, 2021

Related U.S. Application Data
(60) Provisional application No. 62/863,798, filed on Jun. 19, 2019.

(51) Int. Cl. A61N 1/36; A61N 1/05

(52) U.S. Cl. CPC A61N 1/36117; A61N 1/0551; A61N 1/36171; A61N 1/36175; A61N 1/36157; A61N 1/36178; A61N 1/36139

(57) ABSTRACT

This invention relates to devices, methods and substances for use in the treatment of hypertension and/or elevated blood pressure in a subject.

### TECHNICAL FIELD

This invention relates to devices, methods and substances for use in the treatment of hypertension and/or elevated blood pressure in a subject. More specifically, the invention relates to devices, methods and substances for stimulating neural activity in a renal nerve, plexus or neurovascular bundle of a subject for the treatment of hypertension, and/or elevated blood pressure, and/or the treatment of a cardiorespiratory or cardiovascular disorder in a hypertensive subject, and/or the treatment of a cardiorespiratory or cardiovascular disorder in a subject with elevated blood pressure.

### BACKGROUND ART

Hypertension is a global burden, and affects more than 1 in 4 adults in the UK. Persistent hypertension, or high blood pressure, is associated with a number of conditions such as heart disease, stroke, diabetes and kidney disease. In particular, hypertensive patients and patients with elevated blood pressure are at risk of cardiorespiratory disorders such as Chronic Obstructive Pulmonary Disorder (COPD), and cardiovascular disorders such as cardiomyopathy. A number of pharmacological therapies, such as ACE inhibitors, angiotensin-2 receptor blockers, calcium channel blockers and diuretics are currently prescribed to lower blood pressure, however most are concomitant with a litany of side effects.

Although risks that contribute to hypertension and elevated blood pressure are related to lifestyle choices (e.g. diet and exercise), the underlying mechanisms that result in essential (or “primary”) hypertension are largely unknown. It is generally accepted that an overall increase in sympathetic tone is strongly associated with hypertension.

To this end, denervation of nerves supplying the kidney, such as renal nerves, has been touted as a possible therapeutic strategy for the treatment of hypertension. For example, de Jong et al. have showed that denervation of renal nerves reduce hypertensive symptoms that are themselves induced by stimulating renal nerve activity¹. Similarly, others have shown that renal nerve denervation may alleviate resistant hypertension² or hypertension induced by kidney pathology³.

Neural ablation devices have also been designed and tested in clinical trials for denervating renal nerve activity in the treatment of hypertension. However, despite promising initial results⁴,⁵, subsequent studies have failed to show that renal denervation is capable of lowering blood pressure to an extent that is suitable for the treatment of hypertension^{6,7,8}.

Thus, there is a need for identifying further and improved ways of treating hypertension and elevated blood pressure.

### SUMMARY OF THE INVENTION

Accordingly, provided herein are devices, methods and substances for modulating the neural activity of a renal nerve, plexus or neurovascular bundle in a subject for treating hypertension and/or elevated blood pressure. In one embodiment, also provided herein are devices, methods and substances for modulating the neural activity of a renal nerve, or plexus or neurovascular bundle in a subject for treating a cardiorespiratory or cardiovascular disorder in a hypertensive subject, and/or for treating a cardiorespiratory or cardiovascular disorder in a subject with elevated blood pressure. In a preferred embodiment, provided herein are devices, methods and substances for stimulating the neural activity of a renal nerve, plexus or neurovascular bundle in a subject for treating hypertension and/or elevated blood pressure, and/or for treating a cardiorespiratory or cardiovascular disorder in a hypertensive subject, and/or for treating a cardiorespiratory or cardiovascular disorder in a subject with elevated blood pressure. In one embodiment, the cardiorespiratory disorder is Chronic Obstructive Pulmonary Disease (COPD) and/or the cardiovascular disorder is cardiomyopathy.

In a preferred embodiment, the devices, methods and substances of the present invention are for stimulating the neural activity of a renal nerve in a subject.

In particular, provided herein is a device for modulating the neural activity of a renal nerve, plexus or neurovascular bundle of a subject for treating hypertension and/or elevated blood pressure. In one embodiment, provided herein is a device for modulating the neural activity of a renal nerve, plexus or neurovascular bundle of a subject for treating a cardiorespiratory or cardiovascular disorder in a hypertensive subject, and/or for treating a cardiorespiratory or cardiovascular disorder in a subject with elevated blood pressure. In a preferred embodiment, provided herein is a device for stimulating the neural activity of a renal nerve, plexus or neurovascular bundle of a subject for treating hypertension and/or elevated blood pressure, and/or for treating a cardiorespiratory or cardiovascular disorder in a hypertensive subject, and/or for treating a cardiorespiratory or cardiovascular disorder in a subject with elevated blood pressure.

In some embodiments, the invention is useful for treating hypertension. In some embodiments, the invention is useful for treating elevated blood pressure. In some embodiments, the invention is useful for treating cardiorespiratory or cardiovascular disorders in a hypertensive subject. In some embodiments, the invention is useful for treating a cardiorespiratory or cardiovascular disorder in a subject with elevated blood pressure.

In one embodiment, provided herein is a device for stimulating the neural activity of a renal nerve, plexus or neurovascular bundle of a subject for treating hypertension and/or elevated blood pressure, and/or for treating a cardiorespiratory or cardiovascular disorder in a hypertensive subject, and/or for treating a cardiorespiratory or cardiovascular disorder in a subject with elevated blood pressure, the device comprising: one or more electrodes configured to apply an electrical signal to the renal nerve, plexus or neurovascular bundle of the subject; and a controller coupled to the one or more electrodes, the controller controlling the signal to be applied by the one or more electrodes and configured to apply the signal in a periodic cycle such that the signal stimulates the neural activity of the renal nerve, plexus or neurovascular bundle to produce a physiological response in the subject, wherein the physiological response comprises a decrease in mean arterial blood pressure and optionally one or more additional physiological responses selected from the group consisting of: an increase in respiration rate, an increase in diaphragmatic EMG, an increase in hindquarter aortic blood flow, a decrease in renal cortical blood flow, a decrease in systemic vascular resistance and an increase in renal cortical vascular resistance.

In some embodiments of the invention, the stimulation in neural activity is reversible. In some embodiments, the stimulation in neural activity is corrective.

In one embodiment, the signal comprises an alternating current (AC) waveform of from 0.2 Hz to 20 Hz frequency. In a preferred embodiment, the signal comprises an alternating current (AC) wave form of from 1 to 20 Hz, 2 to 20 Hz, 2 to 10 Hz, 2 to 8 Hz, 3 to 7 Hz, 4 Hz to 6 Hz or 5 Hz frequency. In some embodiments, the AC waveform is a sinusoidal waveform.

In one embodiment, the signal comprises a pulse width of 2 ms or less. In a preferred embodiment, the signal comprises a pulse width of from 1 ms to 2 ms.

In one embodiment, the signal comprises a current of 1.0 mA to 30 mA. In a preferred embodiment, the signal comprises a current of 2.0 mA to 30 mA, 5.0 mA to 30 mA, 10 mA to 30 mA, 15 mA to 30 mA, 20 to 30 mA or 15 to 25 mA.

In some embodiments the one or more electrodes are configured to apply a signal in a periodic cycle. In some embodiments, the periodic cycle comprises at least one signal period, wherein the signal is applied during the signal period. In some embodiments, the periodic cycle may comprise up to 12 signal periods within 24 hours. In some embodiments, the periodic cycle may comprise at least two, at least three, at least four, at least five, at least 6, at least 7, at least 8, at least 9, at least ten or at least eleven signal periods within 24 hours. In a preferred embodiment, the periodic cycle comprises at least 4 signal periods within 24 hours. In some embodiments, the signal period has a duration of from 1 second to 2 hours, preferably of about 1 hour.

In some embodiments, the signal is applied continuously during the signal period. In some embodiments, the signal is applied in a burst pattern during the signal period, wherein the burst pattern comprises at least one burst in which the signal is on, and at least one rest period in which the signal is off, wherein the rest period begins at the end of a first burst, and ends at the beginning of a subsequent burst. In some embodiments, each of the at least one bursts and/or rest periods has the same duration. In some embodiments, each of the at least one bursts and/or rest periods has a different duration. In some embodiments, the ratio of the duration of the burst to the duration of the rest period is 1:4, 1:3, 1:2, 1:1, 2:1, 3:1, 4:1, 6:1 or 12:1.

In some embodiments, the signal period has a duration of 1 hour, and the signal is applied continuously for about 2 minutes to about 30 minutes during the signal period. In some embodiments, the signal period has a duration of 1 hour, and the signal is applied in a burst pattern during the signal period, wherein the signal is on for a total of about 2 minutes to about 30 minutes during the signal period.

Where the signal is applied in a burst pattern, the burst preferably has a duration of between 10 and 60 s and the rest period has a duration of between 30 and 120 s. For example, the burst may have duration of ≤10 s, ≤20 s, ≤30 s, ≤40 s, ≤50 s, or ≤60 s. Alternatively or additionally, the duration of the burst may be ≥10 s, ≥20 s, ≥30 s, ≥40 s, ≥50 s, or ≥60 s. Any combination of the upper and lower limits above for the duration of the burst is also possible. The duration of the rest period may be ≤30 s, ≤40 s, ≤50 s, ≤60 s, ≤70 s, ≤80 s, ≤90 s, ≤100 s, ≤110 s, or ≤120 s. Alternatively or additionally, the duration of the rest period may be ≥30 s, ≥40 s, ≥50 s, ≥60 s, ≥70 s, ≥80 s, ≥90 s, ≥100 s, ≥110 s, or ≤120 s. Any combination of the upper and lower limits above for the duration of the rest period is also possible.

In an exemplary embodiment, the burst pattern has a burst duration of 30 s, and a rest period of 90 s.

In some embodiments, the device of the invention is configured to apply the signal for a duration of from 20 to 60 sec. In a preferred embodiment, the device is configured to apply the signal for a duration of 40 sec.

In some embodiments, the periodic cycle may be based on the subject's sleep-wake cycle, in particular the at least one signal period may be whilst the subject is asleep. In some such embodiments, the episodes may be applied between 10 pm and 6 am. The sleep-wake cycle may be measured via known methods by detecting the subject's circadian rhythm phase markers (e.g. cortisol level, melatonin level or core body temperature), and/or a detector for detecting the subject's movements.

In some embodiments, the device of the invention further comprises means to detect one or more physiological parameters in a subject. In some embodiments, the controller is coupled to said means to detect, and causes the one or more electrodes to apply the signal when the physiological parameter is detected to be meeting or exceeding a predefined threshold value. In some embodiments, the one or more detected physiological parameters comprise one or more of mean arterial blood pressure, heart rate, blood flow such as aortic blood flow and/or cortical blood flow, vascular resistance such as aortic vascular resistance and/or cortical vascular resistance, air flow, airway resistance, airway pressure such as upper airway pressure and/or lower airway pressure, diaphragmatic EMG and respiration rate.

In some embodiments, the device of the invention is suitable for at least partial implantation into the subject. In some embodiments, the device is suitable to be wholly implanted into the subject.

Provided herein are methods for treating hypertension in a subject. In one embodiment the hypertension is primary hypertension. In one embodiment, the hypertension is secondary hypertension caused by renal disease. Also provided herein are methods for treating elevated blood pressure in a subject. Also provided herein are methods for treating a cardiorespiratory or cardiovascular disorder in a hypertensive subject. Also provided herein are methods for treating a cardiorespiratory or cardiovascular disorder in a subject with elevated blood pressure. In one embodiment, the method comprises: (i) implanting in the subject a device of the invention; (ii) positioning at least one electrode of the device in signaling contact with a renal nerve, plexus or neurovascular bundle of the subject; and (iii) activating the apparatus to stimulate neural activity of a renal nerve, plexus or neurovascular bundle in the subject.

In some embodiments, renal nerve, plexus or neurovascular bundle stimulation is applied unilaterally or bilaterally. In a preferred embodiment, renal nerve, plexus or neurovascular bundle stimulation is applied bilaterally.

Provided herein is a method of treating hypertension and/or elevated blood pressure in a subject, and/or of treating a cardiorespiratory or cardiovascular disorder in a hypertensive subject, and/or of treating a cardiorespiratory or cardiovascular disorder in a subject with elevated blood pressure, wherein the method comprises applying an electrical signal to a renal nerve, plexus or neurovascular bundle in the subject to stimulate neural activity in the renal nerve, plexus or neurovascular bundle of the subject. In one embodiment, the hypertension is primary hypertension. In one embodiment, the hypertension is secondary hypertension caused by renal disease.

In some embodiments, treatment of hypertension and/or elevated blood pressure is indicated by an improvement in mean arterial blood pressure and optionally an improvement in at least one additional measureable physiological parameter selected from the group consisting of respiration rate, vascular resistance, hindquarter aortic blood flow, cortical blood flow, diaphragmatic EMG and air flow.

In some embodiments, the electrical signal is applied by a neuromodulation device comprising one or more electrodes for applying the electrical signal.

In some embodiments the stimulation in neural activity is reversible. In some embodiments, the stimulation in neural activity is corrective.

In some embodiments signal is applied in a periodic cycle. In some embodiments, the periodic cycle comprises at least one signal period, wherein the signal is applied during the signal period. In some embodiments, the periodic cycle may comprise up to 12 signal periods within 24 hours. In some embodiments, the periodic cycle may comprise at least two, at least three, at least four, at least five, at least 6, at least 7, at least 8, at least 9, at least ten or at least eleven signal periods within 24 hours. In a preferred embodiment, the periodic cycle comprises at least 4 signal periods within 24 hours. In some embodiments, the signal period has a duration of from 1 second to 2 hours, preferably of about 1 hour.

In some embodiments, the signal is applied continuously during the signal period. In some embodiments, the signal is applied in a burst pattern during the signal period, wherein the burst pattern comprises at least one burst in which the signal is on, and at least one rest period in which the signal is off, wherein the rest period begins at the end of a first burst, and ends at the beginning of a subsequent burst. In some embodiments, each of the at least one bursts and/or rest periods has the same duration. In some embodiments, each of the at least one bursts and/or rest periods has a different duration. In some embodiments, the ratio of the duration of the burst to the duration of the rest period is 1:4, 1:3, 1:2, 1:1, 2:1, 3:1, 4:1, 6:1 or 12:1.

In some embodiments, the signal period has a duration of 1 hour, and the signal is applied continuously for about 2 minutes to about 30 minutes during the signal period. In some embodiments, the signal period has a duration of 1 hour, and the signal is applied in a burst pattern during the signal period, wherein the signal is on for a total of about 2 minutes to about 30 minutes during the signal period.

Where the signal is applied in a burst pattern, the burst preferably has a duration of between 10 and 60 s and the rest period has a duration of between 30 and 120 s. For example, the burst may have duration of ≤10 s, ≤20 s, ≤30 s, ≤40 s, ≤50 s, or ≤60 s. Alternatively or additionally, the duration of the burst may be ≥10 s, ≥20 s, ≥30 s, ≥40 s, ≥50 s, or ≥60 s. Any combination of the upper and lower limits above for the duration of the burst is also possible. The duration of the rest period may be ≤30 s, ≤40 s, ≤50 s, ≤60 s, ≤70 s, ≤80 s, ≤90 s, ≤100 s, ≤110 s, or ≤120 s. Alternatively or additionally, the duration of the rest period may be ≥30 s, ≥40 s, ≥50 s, ≥60 s, ≥70 s, ≥80 s, ≥90 s, ≥100 s, ≥110 s, or ≤120 s. Any combination of the upper and lower limits above for the duration of the rest period is also possible.

In an exemplary embodiment, the burst pattern has a burst duration of 30 s, and a rest period of 90 s.

In some embodiments, the device of the invention is configured to apply the signal for a duration of from 20 to 60 sec. In a preferred embodiment, the device is configured to apply the signal for a duration of 40 sec.

In some embodiments, the periodic cycle may be based on the subject's sleep-wake cycle, in particular the at least one signal period may be whilst the subject is asleep. In some such embodiments, the episodes may be applied between 10 pm and 6 am. The sleep-wake cycle may be measured via known methods by detecting the subject's circadian rhythm phase markers (e.g. cortisol level, melatonin level or core body temperature), and/or a detector for detecting the subject's movements.

Also provided herein is a charged particle for use in treating hypertension and/or elevated blood pressure in a subject, and/or for use in treating a cardiorespiratory or cardiovascular disorder in a hypertensive subject, and/or for use in treating a cardiorespiratory or cardiovascular disorder in a subject with elevated blood pressure, wherein the charged particle is administered to a renal nerve, plexus or neurovascular bundle according to a dosage regime defined by an electrical signal having a frequency of from 0.2 to 10 Hz, a pulse width of less than 2 ms and a current of from 1.0 mA to 30.0 mA, wherein the electrical signal is administered in a periodic cycle comprising at least one signal period, in which the signal is applied in a burst pattern comprising at least 4 bursts with a rest period of at least 4 minutes between bursts.

In some embodiments, the charged particle is one or more electrons. In some embodiments, the charged particle is used to treat primary hypertension or secondary hypertension caused by renal disease.

Also provided herein is a computer-implemented method of reversibly stimulating neural activity in a renal nerve, plexus or neurovascular bundle in a subject, the method comprising controlling the operation of at least one electrode of the device of the invention to apply an electrical signal to the renal nerve, plexus or neurovascular bundle to stimulate neural activity. In some embodiments, the computer-implemented method is useful for treating hypertension, such as primary hypertension or secondary hypertension caused by renal disease. In some embodiments, the computer-implemented method is useful for treating a cardiorespiratory or cardiovascular disorder in a subject with elevated blood pressure. In some embodiments, the computer-implemented method is useful for treating a cardiorespiratory or cardiovascular disorder in a hypertensive subject. In some embodiments, the computer-implemented method is useful for treating a cardiorespiratory or cardiovascular disorder in a subject with elevated blood pressure.

Also provided herein is a modulated nerve, plexus or neurovascular bundle to which the device of the invention is in signaling contact, wherein the nerve is a renal nerve, plexus or neurovascular bundle, wherein at least one electrode of the device is in signaling contact with the nerve, plexus or neurovascular bundle so that the nerve, plexus or neurovascular bundle can be distinguished from the nerve in its natural state. In some embodiments, the modulated nerve, plexus or neurovascular bundle is obtainable by stimulating neural activity according to a method of the invention.

Also provided herein is an ACE inhibitor, angiotensin-2 receptor blocker, calcium channel blocker or diuretic for use in the treatment of hypertension, elevated blood pressure, and/or a cardiorespiratory or cardiovascular disorder, in a subject receiving, having received or about to receive the method of the invention.

Also provided herein is an ACE inhibitor, angiotensin-2 receptor blocker, calcium channel blocker or diuretic for use in the treatment of hypertension, elevated blood pressure, and/or a cardiorespiratory or cardiovascular disorder, in a subject implanted with the device of the invention.

### DETAILED DESCRIPTION OF THE INVENTION

Renal Nerve/Plexus Nerves of the autonomous system regulate the kidneys and surrounding tissues (including blood vessels, glomeruli, tubules, etc.) via a rich network of afferent and efferent renal nerves, collectively called the renal plexus. The kidney receives regulatory signals from both the sympathetic and parasympathetic nervous system. Postganglionic nerves innervate arteries supplying the kidneys to regulate blood flow. In patients with hypertension and/or elevated blood pressure, activity in renal sympathetic nerves is commonly increased. Increased activity of the renal sympathetic nerves raises blood pressure, and can also affect proper functioning of the kidneys.

Anatomical mapping of afferent fibres of the rat kidney has identified the pathways traversed by these fibres including locations of their cell bodies in dorsal root ganglia (DRG) of the spinal cord. Afferent nerves arising from the left kidney project ipsilaterally through dorsal roots T8-L2 with most fibres (>90%) confined to dorsal roots T10-L1. Afferent projections arising from the right kidney are found in dorsal roots T9-L1, with most fibres (>90%) in T9-T13. The central projections of many of these renal afferents terminate within the nucleus tractus solitarius (NTS) including medial and caudal subnuclei that control hemodynamic and ventilatory functions, respectively [Ciriello et al., J Auton Nery Syst. 8: 273-285, 1983; Donovan et al., Brain Res. 259: 119-122, 1983; Weiss and Chowdhury, Brain Res. 812: 227-241, 1998; Lappe et al., Am J Physiol. 249: R634-R637, 1985, all of which are incorporated herein by reference]. Viral tracing studies indicate that primary sensory neurons innervating the kidney are found in the dorsal root ganglia in T8-L4 spinal cord levels ipsilateral to the kidney investigated. Kidney infection in the rat with PRV consistently infected cells in four CNS sites: ventrolateral medulla, A5 region of the pons, the caudal raphe nuclei-parapyramidal area and the paraventricular nucleus in the hypothalamus (Weiss and Chowdhury, 1998). Efferent nerves innervate the kidney along the renal artery and vein, and throughout the renal cortex and the outer medulla. Efferent nerves also innervate the vascular smooth muscle cells in the afferent and efferent arterioles and along the inner medulla.

In some embodiments, the invention involves modulating the activity of a renal nerve, preferably a sympathetic renal nerve. In some embodiments, the invention modulates activity of the renal plexus. In some embodiments, the invention modulates activity of a renal neurovascular bundle. In some embodiments, the invention modulates activity of the left renal nerve. In some embodiments, the invention modulates activity of the right renal nerve.

The invention involves modulating neural activity at any site along the renal nerve or its connections to the spinal roots, preferably the dorsal roots, to target renal afferents. For example, the site of stimulation might be along the length of the renal artery where nerves form a plexus, or nerve tracts that innervate the sympathetic ganglion alongside the aorta, most notably the dorsal roots of the spinal cord. Preferably, the site is amenable for attachment of electrodes.

Modulation of Neural Activity

The invention involves modulation of neural activity of a renal nerve, plexus or neurovascular bundle. As used herein, “neural activity” of a nerve means the signaling activity of the nerve, for example the amplitude, frequency and/or pattern of action potentials in the nerve. “Neural activity” may also relate to the activity of a renal plexus and/or neurovascular bundle. The term “pattern”, as used herein in the context of action potentials in the nerve, is intended to include one or more of: local field potential(s), compound action potential(s), aggregate action potential(s), and also magnitudes, frequencies, areas under the curve and other patterns of action potentials in the nerve or sub-groups (e.g. fascicules) of neurons therein.

Modulation of neural activity, as used herein, is taken to mean that the signaling activity of the nerve is altered from the baseline neural activity—that is, the signaling activity of the nerve in the subject prior to any intervention. Modulation may involve creation of action potentials in the nerve compared to baseline activity. The modulation of the nerve according to the present invention results in preferential increased sympathetic signals in the direction of the spinal cord and/or the kidney.

The invention preferentially stimulates the neural activity of the nerve. Stimulation may result in at least part of the nerve being increased compared to baseline neural activity in that part of the nerve. This increase in activity can be across the whole nerve, in which case neural activity is increased across the whole nerve.

Stimulation typically involves increasing neural activity e.g. generating action potentials beyond the point of the stimulation in at least a part of the nerve. At any point along the axon, a functioning nerve will have a distribution of potassium and sodium ions across the nerve membrane. The distribution at one point along the axon determines the electrical membrane potential of the axon at that point, which in turn influences the distribution of potassium and sodium ions at an adjacent point, which in turn determines the electrical membrane potential of the axon at that point, and so on. This is a nerve operating in its normal state, wherein action potentials propagate from point to adjacent point along the axon, and which can be observed using conventional experimentation.

One way of characterizing a stimulation of neural activity is a distribution of potassium and sodium ions at one or more points in the axon, which is created not by virtue of the electrical membrane potential at adjacent a point or points of the nerve as a result of a propagating action potential, but by virtue of the application of a temporary external electrical field. The temporary external electrical field artificially modifies the distribution of potassium and sodium ions within a point in the nerve, causing depolarization of the nerve membrane that would not otherwise occur. The depolarization of the nerve membrane caused by the temporary external electrical field generates de novo action potential across that point. This is a nerve operating in a disrupted state, which can be observed by a distribution of potassium and sodium ions at a point in the axon (the point which has been stimulated) that has an electrical membrane potential that is not influenced or determined by the electrical membrane potential of an adjacent point.

Stimulation of neural activity is thus understood to be increasing neural activity beyond the point of signal application. Thus, the nerve at the point of signal application is modulated in that the nerve membrane is reversibly depolarized by an electric field, such that a de novo action potential is generated and propagates through the modulated nerve. Hence, the nerve at the point of signal application is modulated in that a de novo action potential is generated.

When an electrical signal is used with the invention, the stimulation is based on the influence of electrical currents (e.g. charged particles, which may be one or more electrons in an electrode attached to the nerve, or one or more ions outside the nerve or within the nerve, for instance) on the distribution of ions across the nerve membrane.

Stimulation of neural activity encompasses full stimulation of neural activity in the nerve—that is, embodiments where the total neural activity is increased in the whole nerve.

Stimulation of neural activity may be partial stimulation. Partial stimulation may be such that the total signaling activity of the whole nerve is partially increased, or that the total signaling activity of a subset of nerve fibers of the nerve is fully increased (i.e. there is only neural activity in that subset of fibers of the nerve), or that the total signaling of a subset of nerve fibers of the nerve is partially increased compared to baseline neural activity in that subset of fibers of the nerve. For example, an increase in neural activity of ≤5%, ≤10%, ≤15%, ≤20%, ≤25%, ≤30%, ≤35%, ≤40%, ≤45%, ≤50%, ≤60%, ≤70%, ≤80%, ≤90% or ≤95%, or an increase of neural activity in a subset of nerve fibers of the nerve. The neural activity may be measured by methods known in the art, for example, by the number of action potentials which propagate through the axon and/or the amplitude of the local field potential reflecting the summed activity of the action potentials.

Modulation of neural activity may be an alteration in the pattern of action potentials. It will be appreciated that the pattern of action potentials can be modulated without necessarily changing the overall frequency or amplitude. For example, modulation of neural activity may be such that the pattern of action potentials is altered to more closely resemble a healthy state rather than a disease state.

Modulation of neural activity may comprise altering the neural activity in various other ways, for example increasing or decreasing a particular part of the neural activity and/or stimulating new elements of activity, for example: in particular intervals of time, in particular frequency bands, according to particular patterns and so forth.

Modulation of neural activity may be (at least partially) corrective. As used herein, “corrective” is taken to mean that the modulated neural activity alters the neural activity towards the pattern of neural activity in a healthy subject, and this is called axonal modulation therapy. That is, upon cessation of signal application, neural activity in the nerve more closely resembles (ideally, substantially fully resembles) the pattern of action potentials in the nerve observed in a healthy subject than prior to signal application. Such corrective modulation can be any modulation as defined herein. For example, application of a signal may result in an increase on neural activity, and upon cessation of signal application the pattern of action potentials in the nerve resembles the pattern of action potentials observed in a healthy subject. By way of further example, application of the signal may result in neural activity resembling the pattern of action potentials observed in a healthy subject and, upon cessation of the signal, the pattern of action potentials in the nerve remains the pattern of action potentials observed in a healthy subject. In some embodiments of the present invention, a device is provided wherein the stimulation in neural activity is corrective.

One advantage of the invention is that modulation of neural activity is reversible. Hence, the modulation of neural activity is not permanent. For example, upon cessation of the application of a signal, neural activity in the nerve returns substantially towards baseline neural activity within 1-60 seconds, or within 1-60 minutes, or within 1-24 hours (e.g. within 1-12 hours, 1-6 hours, 1-4 hours, 1-2 hours), or within 1-7 days (e.g. 1-4 days, 1-2 days). In some instances of reversible modulation, the neural activity returns substantially fully to baseline neural activity. That is, the neural activity following cessation of the application of a signal is substantially the same as the neural activity prior to a signal being applied. Hence, the nerve or the portion of the nerve has regained its normal physiological capacity to propagate action potentials. In some embodiments of the present invention, a device is provided wherein the stimulation in neural activity is reversible.

In other embodiments, modulation of the neural activity may be substantially persistent. As used herein, “persistent” is taken to mean that the modulated neural activity has a prolonged effect. For example, upon cessation of the application of a signal, neural activity in the nerve remains substantially the same as when the signal was being applied—i.e. the neural activity during and following signal application is substantially the same. Reversible modulation is preferred.

Hypertension and Elevated Blood Pressure

The invention is useful for treating hypertension and/or elevated blood pressure. In some embodiments, the invention is useful for treating cardiorespiratory or cardiovascular disorders in a hypertensive subject. In some embodiments, the invention is useful for treating a cardiorespiratory or cardiovascular disorder in a subject with elevated blood pressure. In some embodiments, the cardiorespiratory disorder is Chronic Obstructive Pulmonary Disease (COPD). In some embodiments, the cardiovascular disorder is cardiomyopathy. Hypertension is characterized by persistently elevated blood pressure, either systolic or diastolic, compared to the normal physiological state. In some embodiments, elevated blood pressure is characterized by systolic blood pressure higher than 120 mmHg, when measured at least twice per hour during the person's usual waking hours, over a 24 hour period. In some embodiments, hypertension is characterized by systolic blood pressure higher than 130 mmHg when measured at least twice per hour during the person's usual waking hours, over a 24 hour period. In some embodiments, elevated blood pressure is characterized by blood pressure higher than 120/80 mmHg when measured at least twice per hour during the person's usual waking hours, over a 24 hour period. In some embodiments, hypertension is characterized by blood pressure higher than 130/80 mmHg when measured at least twice per hour during the person's usual waking hours, over a 24 hour period. However, blood pressure measurements can vary between hypertensive patients. Blood pressure measurements can also vary between patients with elevated blood pressure.

Hypertension can be primary or secondary (caused by another condition). In one embodiment, the invention is useful for treating primary hypertension. In one embodiment, the invention is useful for treating secondary hypertension, preferably secondary hypertension caused by renal disease.

The invention is useful for treating subjects suffering from, or are at risk in developing, hypertension. The invention is also useful for treating subjects suffering from, or are at risk in developing, elevated blood pressure. The invention may treat or ameliorate the effects of hypertension and/or elevated blood pressure by reducing blood pressure. This may be achieved by modulating physiological functions playing important roles in hypertension and/or elevated blood pressure by reversibly electrically stimulating neural activity in the renal nerve, plexus or neurovascular bundle. The invention induces improvements in a range of haemodynamic, kidney and respiratory associated functions.

In some embodiments, the invention is useful for modulating renal haemodynamic control. In some embodiments, the invention is useful for modulating systemic haemodynamic control.

Treatment of hypertension and/or elevated blood pressure can be assessed in various ways, but typically involves determining an improvement in one or more physiological parameters of the subject.

Useful physiological parameters of the invention may be one or more of the group consisting of: mean arterial blood pressure, heart rate, blood flow such as aortic blood flow and/or cortical blood flow, vascular resistance such as aortic vascular resistance and/or cortical vascular resistance, air flow, airway resistance, airway pressure such as upper airway pressure and/or lower airway pressure, diaphragmatic EMG and respiration rate

In some embodiments, the invention modulates cardiovascular function. In some embodiments, the invention modulates central neuroregulation of respiration.

As used herein, an “improvement in a determined physiological parameter” is taken to mean that, for any given physiological parameter, an improvement is a change in the value of that parameter in the subject towards the normal value or normal range for that value—i.e. towards the expected value in a healthy subject. As used herein, “worsening of a determined physiological parameter” is taken to mean that, for any given physiological parameter, worsening is a change in the value of that parameter in the subject away from the normal value or normal range for that value—i.e. away from the expected value in a healthy subject.

Improvement in a determined physiological parameter according to the invention is indicated by one or more of the group consisting of: a decrease in mean arterial blood pressure, a decrease in heart rate, an increase in blood flow such as aortic blood flow and/or a decrease in cortical blood flow, a decrease in vascular resistance such as aortic vascular resistance and/or an increase in cortical vascular resistance, an increase in air flow, a decrease in airway resistance, and/or an increase in diaphragmatic EMG. The invention might not lead to a change in all of these parameters.

As used herein, a physiological parameter is not affected by the modulation (e.g. stimulation) of the neural activity if the parameter does not change (in response to nerve modulation) from the normal value or normal range for that value of that parameter exhibited by the subject or subject when no intervention has been performed i.e. it does not depart from the baseline value for that parameter. Suitable methods for determining changes in any of these physiological parameters would be appreciated by the skilled person.

The skilled person will appreciate that the baseline for any neural activity or physiological parameter in a subject need not be a fixed or specific value, but rather can fluctuate within a normal range or may be an average value with associated error and confidence intervals. Suitable methods for determining baseline values are well known to the skilled person.

As used herein, a physiological parameter is determined in a subject when the value for that parameter exhibited by the subject at the time of detection is determined. A detector (e.g. a physiological sensor subsystem, a physiological data processing module, a physiological sensor, etc.) is any element able to make such a determination.

Thus, in certain embodiments, the invention further comprises a step of determining one or more physiological parameters of the subject, wherein the signal is applied only when the determined physiological parameter meets or exceeds a predefined threshold value. In such embodiments wherein more than one physiological parameter of the subject is determined, the signal may be applied when any one of the determined physiological parameters meets or exceeds its threshold value, alternatively only when all of the determined physiological parameters meet or exceed their threshold values. In certain embodiments wherein the signal is applied by a system of the invention, the system further comprises at least one detector configured to determine the one or more physiological parameters of the subject.

In certain embodiments, the physiological parameter is an action potential or pattern of action potentials in a nerve of the subject, wherein the action potential or pattern of action potentials is associated with the condition that is to be treated.

It will be appreciated that any two physiological parameters may be determined in parallel embodiments, the controller is coupled to detect the pattern of action potentials tolerance in the subject.

A predefined threshold value for a physiological parameter is the minimum (or maximum) value for that parameter that must be exhibited by a subject or subject before the specified intervention is applied. For any given parameter, the threshold value may be defined as a value indicative of a pathological state or a disease state. The threshold value may be defined as a value indicative of the onset of a pathological state or a disease state. Thus, depending on the predefined threshold value, the invention can be used as a treatment. Alternatively, the threshold value may be defined as a value indicative of a physiological state of the subject (that the subject is, for example, asleep, post-prandial, or exercising). Appropriate values for any given physiological parameter would be simply determined by the skilled person (for example, with reference to medical standards of practice).

Such a threshold value for a given physiological parameter is exceeded if the value exhibited by the subject is beyond the threshold value—that is, the exhibited value is a greater departure from the normal or healthy value for that physiological parameter than the predefined threshold value.

A subject of the invention may, in addition to having a system according to the invention, receive medicine for their condition. For instance, a subject having a device according to the invention may receive an anti-hypertensive medicine. Such medicines include ACE inhibitors, angiotensin-2 receptor blockers, calcium channel blockers and diuretics. Thus the invention provides the use of these medicines in combination with a device of the invention.

A System for Implementing the Invention

A system according to the invention comprises a device, the device may be implantable (e.g. implantable device 106 of FIG. 1). In some embodiments of the invention, the device is suitable for at least partial implantation into the subject, optionally suitable to be wholly implanted into the subject. The implantable device comprises at least one neural interface 108 comprising at least one electrode (e.g. electrode 109), suitable for placement on or around a renal nerve, plexus or neurovascular bundle. In some embodiments, the neural interface is implanted unilaterally on the renal nerve, plexus or neurovascular bundle. In some embodiments, the neural interface is implanted bilaterally on the renal nerve, plexus or neurovascular bundle. The system preferably also comprises a processor (e.g. microprocessor 113) coupled to the at least one electrode.

The at least one electrode may take many forms, and includes any component which, when used in an implantable system for implementing the invention, is capable of applying a stimulus or other signal that modulates electrical activity, e.g., action potentials, in a nerve.

The various components of the system are preferably part of a single physical device, either sharing a common housing or being a physically separated collection of interconnected components connected by electrical leads (e.g. leads 107). As an alternative, however, the invention may use a system in which the components are physically separate, and communicate wirelessly. Thus, for instance, the at least one electrode (e.g. electrode 109) and the implantable device (e.g. implantable device 106) can be part of a unitary device, or together may form a system (e.g. system 116). In both cases, further components may also be present to form a wider system (e.g. system 100).

Suitable Forms of a Signal

The invention uses a signal applied via one or more electrode (e.g. electrode 109) placed in signaling contact with a renal nerve, renal plexus, or neurovascular bundle preferably on or around the nerve, plexus or neurovascular bundle.

Signals applied according to the invention are ideally non-destructive. As used herein, a “non-destructive signal” is a signal that, when applied, does not irreversibly damage the underlying neural signal conduction ability of the nerve. That is, application of a non-destructive signal maintains the ability of the nerve or fibers thereof, or other nerve tissue to which the signal is applied, to conduct action potentials when application of the signal ceases, even if that conduction is in practice artificially stimulated as a result of application of the non-destructive signal.

The signal will usually be an electrical signal, which may be, for example, a voltage or current waveform. The at least one electrode (e.g. electrode 109) of the system (e.g. system 116) is configured to apply the electrical signals to a nerve, or a part thereof. However, electrical signals are just one way of implementing the invention, as is further discussed below.

An electrical signal can take various forms, for example, a voltage or current. In certain such embodiments the signal applied comprises a direct current (DC), such as a charge-balanced DC, or a charged-balance alternating current (AC) waveform, or both a DC and an AC waveform. As used herein, “charge-balanced” in relation to a DC current is taken to mean that the positive or negative charge introduced into any system (e.g. a nerve) as a result of a DC current being applied is balanced by the introduction of the opposite charge in order to achieve overall (net) neutrality. In other words, a charge-balance DC current includes a cathodic pulse and an anodic pulse.

In some embodiments, the signal is applied unilaterally. In some embodiments, the signal is applied bilaterally.

The signal may be applied in a periodic cycle comprising at least one signal period. As used herein ‘signal period’ means a period of time during which the electrical signal of the invention is applied. The periodic cycle may comprise at least 1, at least 2, at least 3, at least 4, at least 5, at least 6, at least 8, or at least 10 signal periods within 24 hours. In a preferred embodiment, the periodic cycle comprises at least 4 signal periods within 24 hours. The signal period may have a duration of from 1 second to 2 hours. In a preferred embodiment, the signal period has a duration of about 1 hour.

In some embodiments, the signal is applied continuously during the signal period. As used herein ‘continuously’ means that the signal is applied without disruption. In some embodiments, the signal is applied in a burst pattern during the signal period, wherein the burst pattern comprises at least one burst in which the signal is on, and at least one rest period in which the signal is off, wherein the rest period begins at the end of a first burst, and ends at the beginning of a subsequent burst. In some embodiments, each of the at least one bursts may have the same or different signal parameters, such as frequency, amplitude, pulse width and/or duration. It will be appreciated that a signal applied during a burst may comprise one or more pulses (i.e. a pulse train), wherein each pulse has a pulse width as herein described. Pulse waveforms such as square, sawtooth, sinusoidal, triangular, trapezoidal, quasitrapezoidal or complex waveforms may be applied during a burst. Therefore, as used herein, when a signal is ‘on’ during a burst, the pulse train may comprise periods between pulses when the signal is not applied. In other words, when the signal is applied in a pulse train, the gaps between those pulses (i.e. between the pulse width and the phase duration) do not mean the signal is not ‘on’.

In some embodiments, each of the at least one bursts and/or rest periods has the same duration. In some embodiments, each of the at least one bursts has a different duration. In some embodiments, the ratio of the duration of the burst to the duration of the rest period is 1:4, 1:3, 1:2, 1:1, 2:1, 3:1, 4:1, 6:1 or 12:1.

The burst preferably has a duration of between 10 and 60 s and the rest period has a duration of between 30 and 120 s. For example, the burst may have duration of ≤10 s, ≤20 s, ≤30 s, ≤40 s, ≤50 s, or ≤60 s. Alternatively or additionally, the on duration may be ≥10 s, ≥20 s, ≥30 s, ≥40 s, ≥50 s, or ≥60 s. Any combination of the upper and lower limits above for the duration of the burst is also possible. For example, the duration of the rest period may be ≤30 s, ≤40 s, ≤50 s, ≤60 s, ≤70 s, ≤80 s, ≤90 s, ≤100 s, ≤110 s, or ≤120 s. Alternatively or additionally, the off duration may be ≥30 s, ≥40 s, ≥50 s, ≥60 s, ≥70 s, ≥80 s, ≥90 s, ≥100 s, ≥110 s, or ≤120 s. Any combination of the upper and lower limits above for the duration of the rest period is also possible.

In an exemplary embodiment, the burst pattern has a burst duration of 30 s, and a rest period of 90 s.

In some embodiments, a periodic cycle may contain one or more signal periods in which the signal is applied continuously and one or more periods in which the signal is applied in a burst pattern. The signal periods within a periodic cycle may have the same or different burst patterns. The signals applied during the one or more signal periods may have the same or different signal parameters, such as frequency, amplitude, pulse width and/or duration. In some embodiments, each of the at least one signal periods has the same duration. In some embodiments, each of the at least one signal periods has a different duration.

In some embodiments, the signal period has a duration of 1 hour, and the signal is applied continuously for about 2 minutes to about 30 minutes during the signal period. In some embodiments, the signal period has a duration of 1 hour, and the signal is applied in a burst pattern during the signal period, wherein the signal is on for a total of about 2 minutes to about 30 minutes during the signal period.

The electric signal may be applied with a step change or with a ramp change in current or intensity. Particular signal parameters for modulating (e.g. stimulating) a renal nerve are further described below.

Modulation of the neural activity of the nerve can be achieved using electrical signals which serve to replicate the normal neural activity of the nerve.

With reference again to FIG. 1, the system 116 comprises an implantable device 106 which may comprise a signal generator 117; for example, a pulse generator. When the implantable device comprises a pulse generator, the implantable device 106 may be referred to as an implantable pulse generator. The signal generator 117 may also be a voltage or current source. The signal generator 117 may be pre-programmed to deliver one or more pre-defined waveforms with signal parameters falling within the range given below. Alternatively, the signal generator 117 may be controllable to adjust one or more of the signal parameters described further below. Control may be open loop, wherein the operator of the implantable device 106 may configure the signal generator using an external controller (e.g. controller 101), or control may be closed loop, wherein signal generator modifies the signal parameters in response to one or more physiological parameters of the subject, as is further described below.

Signal Parameters

In all of the above examples, the signal generator 117 may be configured to deliver an electrical signal to modulate (e.g. stimulate) a renal nerve, plexus or neurovascular bundle. For example, the signal generator 117 is configured to apply an electrical signal with certain electrical signal parameters to stimulate neural activity in the nerve, plexus or neurovascular bundle. Signal parameters for stimulating neural activity in the nerve, which are described in detail below, may include waveform, amplitude, and frequency.

The signal parameters described herein are applicable independently to the signal to be applied to a renal nerve, plexus or neurovascular bundle.

Waveform

Modulation (e.g. stimulation) of a renal nerve, plexus or neurovascular bundle can be achieved using electrical signals which serve to replicate the normal neural activity of the nerve. Thus, the waveform of the electrical signal comprises one or more pulse trains, each with a defined pulse width. Pulse waveforms such as square, sawtooth, sinusoidal, triangular, trapezoidal, quasitrapezodial or complex waveforms may be used with the invention. In one preferred embodiment, the waveform is an AC sinusoidal waveform.

In some embodiments, the signal comprises a pulse width between 1 μs and 2 ms, such as between 1 ms and 2 ms or between 1 μs and 1 ms. In some embodiments, the signal comprises a pulse width of 2 ms or less. In one embodiment, the signal comprises a pulse width of from 0.4 ms to 2 ms. In a preferred embodiment, the signal comprises a pulse width of from 1 ms to 2 ms. In some embodiments, the pulses have a pulse width of from 0.6 to 2 ms, 0.8 to 2 ms, 1.2 ms to 1.8 ms, 1.3 ms to 1.7 ms, 1.4 ms to 1.6 ms or 1.5 ms (including, if applicable, both positive and negative phases of the pulse, in the case of a charge-balanced biphasic pulse). In some embodiments, the pulse width may be ≤2 ms, ≤1.8 ms, ≤1.5 ms, ≤1.2 ms, ≤1 ms, ≤0.8 ms, ≤0.5 ms or ≤0.2 ms. Additionally or alternatively, the pulse width may be ≥2 ms, ≥1.8 ms, ≥1.5 ms, ≥1.2 ms, ≥1 ms, ≥0.8 ms, ≥0.5 ms, ≥0.2 ms or ≥1 μs. Any combination of the upper and lower limits above is also possible. The pulse width may additionally be limited by the frequency. In a preferred embodiment, the signal comprises a pulse width of from 1 ms to 2 ms.

The pulses may be charge-balanced. A charge-balanced pulse refers to a pulse which, over the period of the pulse, applies equal amounts (or thereabouts) of positive and negative charge to the nerve.

In some embodiments, the pulses are biphasic pulses. The term “biphasic” refers to a signal which delivers to the nerve over time both a positive and negative charge. The biphasic pulses are preferably charge-balanced. The term “charge-balanced” in relation to a pulse train is taken to mean that the positive charge and negative charge applied by the signal over the pulse duration is equal. In other embodiments, the pulses are monophasic pulses.

The pulses may be symmetric or asymmetric. A symmetric pulse is a pulse where the waveform when applying a positive charge to the nerve is symmetrical to the waveform when applying a negative charge to the nerve. An asymmetric pulse is a pulse where the waveform when applying a positive charge to the nerve is not symmetrical with the waveform when applying a negative charge to the nerve.

In some embodiments, the pulses are biphasic square pulses. In an exemplary embodiment, the waveform is a pulse train with biphasic, asymmetric square pulses.

Amplitude

The electrical signal may have an amplitude between 1.0 mA to 30 mA, e.g. between 2.0 mA and 30 mA, or between 5.0 mA and 30 mA, or between 10 mA and 30 mA, or between 15 mA and 30 mA, or between 20 and 30 mA, or between 15 and 25 mA. In some embodiments, the amplitude may be ≤30 mA, ≤29 mA, ≤28 mA, ≤27 mA, ≤26 mA, ≤25 mA, ≤24 mA, ≤22 mA, ≤20 mA, ≤15 mA, ≤10 mA, or ≤5 mA. In some embodiment, the amplitude may be ≥29 mA, ≥28 mA, ≥27 mA, ≥26 mA, ≥25 mA, ≥24 mA, ≥22 mA, ≥20 mA, ≥15 mA, ≥10 mA, ≥5 mA, or ≥2 mA. Any combination of the upper and lower limits is also possible. In a preferred embodiment, the electrical signal has an amplitude between 10 mA and 30 mA.

It would be of course understood in the art that the electrical signal applied to the nerve would be within clinical safety margins (e.g. suitable for maintaining nerve signaling function, suitable for maintaining nerve integrity, and suitable for maintaining the safety of the subject). The electrical parameters within the clinical safety margin would typically be determined by pre-clinical studies.

It will be appreciated by the skilled person that the current amplitude of an applied electrical signal necessary to achieve the intended modulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject.

The burst preferably has a duration of between 10 and 60 s and the rest period has a duration of between 30 and 120 s. For example, the burst may have duration of ≤10 s, ≤20 s, ≤30 s, ≤40 s, ≤50 s, or ≤60 s. Alternatively or additionally, the on duration may be ≥10 s, ≥20 s, ≥30 s, ≥40 s, ≥50 s, or ≥60 s. Any combination of the upper and lower limits above for the duration of the burst is also possible. For example, the duration of the rest period may be ≤30 s, ≤40 s, ≤50 s, ≤60 s, ≤70 s, ≤80 s, ≤90 s, ≤100 s, ≤110 s, or ≤120 s. Alternatively or additionally, the off duration may be ≥30 s, ≥40 s, ≥50 s, ≥60 s, ≥70 s, ≥80 s, ≥90 s, ≥100 s, ≥110 s, or ≤120 s. Any combination of the upper and lower limits above for the duration of the rest period is also possible.

In an exemplary embodiment, the burst pattern has a burst duration of 30 s, and a rest period of 90 s.

In some embodiments, the device of the invention is configured to apply the signal for a duration of from 20 to 60 sec. In a preferred embodiment, the device is configured to apply the signal for a duration of 40 sec.

In some embodiments, the periodic cycle may be based on the subject's sleep-wake cycle, in particular the at least one signal period may be whilst the subject is asleep. In some such embodiments, the episodes may be applied between 10 pm and 6 am. The sleep-wake cycle may be measured via known methods by detecting the subject's circadian rhythm phase markers (e.g. cortisol level, melatonin level or core body temperature), and/or a detector for detecting the subject's movements.

Frequency

Frequency is defined as the reciprocal of the phase duration of the electrical waveform (i.e. 1/phase).

The inventors have found preferred frequencies for stimulating a renal nerve, plexus or neurovascular bundle. In particular, the inventors have found preferred frequencies for embodiments where the electrical signal is applied periodically and for embodiments where the electrical signal is applied continuously.

In certain embodiments, the electrical signal has a frequency of from 0.2 Hz to 20 Hz, e.g. from 1 to 20 Hz, from 2 to 20 Hz, from 2 to 10 Hz, from 2 to 8 Hz, from 3 to 7 Hz, from 4 Hz to 6 Hz, or 5 Hz frequency. In a preferred embodiment, the electrical signal has a frequency of about 5 Hz. In some embodiments, the frequency of the electrical signal may be ≤20 Hz, ≤15 Hz, ≤10 Hz, ≤8 Hz, ≤7 Hz, ≤6 Hz, ≤5 Hz, ≤4 Hz, ≤3 Hz, ≤2 Hz, or ≤1 Hz. Additionally or alternatively, the frequency of the electrical signal may be ≥0.2 Hz, ≥1 Hz, ≥2 Hz, ≥3 Hz, ≥4 Hz, ≥5 Hz ≥6 Hz, ≥7 Hz, ≥8 Hz ≥9 Hz, ≥10 Hz, ≥15 Hz or ≥20 Hz. Any combination of the upper and lower limits above is also possible.

The signal generator 117 may be configured to deliver one or more pulse trains at intervals according to the above-mentioned frequencies. For example, a frequency of 1 to 50 Hz results in a pulse interval between 1 pulse per second and 50 pulses per second, within a given pulse train.

Geometry of the Electrodes

As explained above, the system comprises at least one electrode. In some embodiments, the one or more electrodes are positioned on at least one neural interface. The at least one neural interface and/or electrode is configured to at least partially circumvent the nerve, plexus or neurovascular bundle and may fully circumvent the nerve, plexus or neurovascular bundle.

In some embodiments, the neural interface forms a cuff around the nerve, plexus or neurovascular bundle (e.g. spiral cuff, helical cuff or flat interface). In other embodiments, the neural interface is a patch. In further embodiments, the neural interface is a clip comprising a first jaw pivoted at one end to a second jaw, and a biasing means (e.g. a mechanical spring) to bias the first and second jaw together.

The geometry of the at least one neural interface and/or electrode is defined in part by the anatomy of the nerve, plexus or neurovascular bundle according to the invention. For example, the geometry of the neural interface and/or the electrode may be limited by the length of the nerve, plexus or neurovascular bundle and/or by the diameter of the nerve, plexus or neurovascular bundle.

In some embodiments, a renal nerve, plexus or neurovascular bundle may be modulated by a neural interface and/or electrode that is suitable for placement on or around the renal nerve, plexus or neurovascular bundle. In some embodiments, the neural interface is placed unilaterally on the renal nerve. In some embodiments, the neural interface is placed bilaterally on the renal nerve.

There may be a plurality of neural interfaces, each neural interface with at least one electrode to define multiple sites for applying a signal. In some embodiments, the multiple sites may be located along a single renal nerve. In other embodiments, the multiple sites may be located on more than one nerve.

Electrode

Electrode types suitable for the present invention are known in the art. For example, [9] disclose several types of electrode for non-damaging neural tissue modulation. The document discloses cuff electrodes (e.g. spiral cuff, helical cuff or flat interface), and flat interface electrodes, both of which are also suitable for use with the present invention. Thus, in one embodiment the electrode is a cuff electrode. In one embodiment the electrode is a flat interface electrode. A mesh, a linear rod-shaped lead, paddle-style lead or disc contact electrode (including multi-disc contact electrodes) are also disclosed in [9] and would be suitable for use in the present invention. A hook electrode, such as a hook electrode from Harvard Apparatus (Holliston, USA), is useful for acute electrical stimulation. A bipolar electrode, such as a bipolar electrode from Cortec (Freiburg, Germany), is useful for chronic implantation. A sling electrode also suitable for the present invention. Also suitable for the present invention are intrafascicular electrode, glass suction electrode, paddle electrode, bipolar hemi-cuff electrode, bipolar hook electrode, percutaneous cylindrical electrode.

Electrodes may be monopolar, bipolar, tripolar, quadripolar or have five or more poles. The electrodes may fabricated from, or be partially or entirely coated with, a high charge capacity material such as platinum black, iridium oxide, titanium nitride, tantalum, poly(elthylenedioxythiophene) and suitable combinations thereof.

In some embodiments, a plurality of electrodes may be positioned at a single site for applying a signal. For example, there may be two or three electrodes for applying a signal. In such embodiments, the electrodes may be positioned on a neural interface such that, in use, the electrodes are located transversely along the axis of the nerve. The surface area of the electrode which is in contact with the nerve may be approximately equal for each electrode.

The plurality of electrodes at a single site may be insulated from one another by a non-conductive biocompatible material. To this end, the neural interface 108 may comprise a non-conductive biocompatible material which is spaced transversely along the nerve when the device is in use.

In some embodiments, each of the plurality of electrodes may be individually electrically excitable. In these embodiments, the signal generator is electrically connected to each electrode separately via one of a plurality of electrical leads, or by any other method known in the art. The signal generator, or a plurality of signal generators, may then apply a different electrical signal to each of the plurality of electrodes. In some instances, no electrical signal may be applied to some of the plurality of electrodes.

Reference [20] discloses separated-interface nerve electrodes, and in particular forms of ionic coupling electrodes (for example in the form of a cuff electrode) that facilitates the application of a prolonged single phase current to a nerve which mitigates the kind of nerve damage described elsewhere herein. This kind of electrode would be suitable for use in the present invention.

In some embodiments (for example, FIG. 1), at least one electrode 109 may be coupled to implantable device 106 of system 116 via electrical leads 107. Alternatively, implantable device 106 may be directly integrated with the at least one electrode 109 without leads. In any case, implantable device 106 may comprise DC current blocking output circuits, optionally based on capacitors and/or inductors, on all output channels (e.g. outputs to the at least one electrode 109, or physiological sensor 111).

Controller

The device of the invention may comprise a controller. In some embodiments, the controller may comprise a processor, for example microprocessor 113. Microprocessor 113 may be responsible for triggering the beginning and/or end of the signals delivered to the nerve, plexus or neurovascular bundle by the at least one electrode. Optionally, microprocessor 113 may also be responsible for generating and/or controlling the signal parameters.

Microprocessor 113 may be configured to operate in an open-loop fashion, wherein a pre-defined signal (e.g. as described above) is delivered to the nerve, plexus or neurovascular bundle at a given periodicity (or continuously) and for a given duration (or indefinitely) with or without an external trigger, and without any control or feedback mechanism. Alternatively, microprocessor 113 may be configured to operate in a closed-loop fashion, wherein a signal is applied based on a control or feedback mechanism. As described elsewhere herein, the external trigger may be an external controller 101 operable by the operator to initiate delivery of a signal.

Microprocessor 113 of the system 116, in particular of the implantable device 106, is preferably constructed so as to generate, in use, a preconfigured and/or operator-selectable signal that is independent of any input. In other embodiments, microprocessor 113 is responsive to an external signal, more preferably information (e.g. data) pertaining to one or more physiological parameters of the subject.

Microprocessor 113 may be triggered upon receipt of a signal generated by an operator, such as a physician or the subject in which the device 106 is implanted. To that end, the system 116 may be part of a system 100 which additionally comprises an external system 118 comprising a controller 101. An example of such a system is described below with reference to FIG. 1.

External system 118 of wider system 100 is external to the system 116 and external to the subject, and comprises controller 101. Controller 101 may be used for controlling and/or externally powering system 116. To this end, controller 101 may comprise a powering unit 102 and/or a programming unit 103. The external system 118 may further comprise a power transmission antenna 104 and a data transmission antenna 105, as further described below.

In one embodiment, the controller controls the signal to be applied by the one or more electrodes. The controller 101 and/or microprocessor 113 may be configured to apply any one or more of the above signals to the nerve, plexus or neurovascular bundle continuously, and/or in a burst pattern. In a preferred embodiment, the controller is configured to apply the signal in a periodic cycle, preferably such that the signal stimulates the neural activity of the renal nerve to produce a physiological response in the subject.

‘Periodic cycle’ refers to where the electrical signal is applied to the nerve for a discrete number of signal periods throughout a day. In some embodiments, the signal is preferably applied for a maximum of 12 signal periods per day.

Each signal period may be defined by a certain duration and/or a certain number of iterations of the electrical signal.

In some embodiments, e.g. where a high frequency signal such as ≥5 Hz is used, the preferred duration for application of the signal to the nerve is less than 10 min, and more preferably between 2 and 5 min.

For example, the signal may be applied for one of: ≤2 min 30 sec, ≤3 min, ≤30 min 30 sec, ≤4 min, ≤4 min 30 sec, or ≤5 min. Alternatively or additionally, the signal may be applied for one of: ≥2 min, ≥2 min 30 sec, ≥3 min, ≥30 min 30 sec, ≥4 min, or ≥4 min 30 sec.

In other embodiments, e.g. where a low frequency signal such as ≤5 Hz is used, the preferred duration for application of the signal to the nerve is less than 2 hours. For example, the signal may be applied for one of: ≤30 min, ≤45 min, ≤1 hour, ≤1 hour 15 min, ≤1 hour 30 min, ≤1 hour 45 min, or ≤2 hours. Alternatively or additionally, the signal may be applied for one of: ≥15 min, ≥30 min, ≥45 min, ≥1 hour, ≥1 hour 15 min, ≥1 hour 30 min, or ≥1 hour 45 min.

Continuous application refers to where the electrical signal is applied to the nerve in a continuous manner. Where the electrical signal is applied continuously within a signal period, it means that the signal is applied in a continuous manner for each signal period of application. In embodiments where the electrical signal is a series of pulses, the gaps between those pulses (i.e. between the pulse width and the phase duration) do not mean the signal is not continuously applied.

Continuous application may continue indefinitely, e.g. permanently. Alternatively, the continuous application may be for a minimum period, for example the signal may be continuously applied for at least 5 days, or at least 7 days.

When the signal is applied in a burst pattern, each burst preferably has a duration of between 10 and 60 s and the rest period has a duration of between 30 and 120 s. For example, the burst may have duration of ≤10 s, ≤20 s, ≤30 s, ≤40 s, ≤50 s, or ≤60 s. Alternatively or additionally, the on duration may be ≥10 s, ≥20 s, ≥30 s, ≥40 s, ≥50 s, or ≥60 s. Any combination of the upper and lower limits above for the duration of the burst is also possible. For example, the duration of the rest period may be ≤30 s, ≤40 s, ≤50 s, ≤60 s, ≤70 s, ≤80 s, ≤90 s, ≤100 s, ≤110 s, or ≤120 s. Alternatively or additionally, the off duration may be ≥30 s, ≥40 s, ≥50 s, ≥60 s, ≥70 s, ≥80 s, ≥90 s, ≥100 s, ≥110 s, or ≤120 s. Any combination of the upper and lower limits above for the duration of the rest period is also possible.

In an exemplary embodiment, the burst pattern has a burst duration of 30 s, and a rest period of 90 s.

In some embodiments, a periodic cycle may contain one or more signal periods in which the signal is applied continuously and one or more periods in which the signal is applied in a burst pattern. The signal periods within a periodic cycle may have the same or different burst patterns. The signals applied during the one or more signal periods may have the same or different signal parameters, such as frequency, amplitude, pulse width and/or duration. In some embodiments, each of the at least one signal periods has the same duration. In some embodiments, each of the at least one signal periods has a different duration.

In some embodiments, the signal period has a duration of 1 hour, and the signal is applied continuously for about 2 minutes to about 30 minutes during the signal period. In some embodiments, the signal period has a duration of 1 hour, and the signal is applied in a burst pattern during the signal period, wherein the signal is on for a total of about 2 minutes to about 30 minutes during the signal period.

In certain embodiments, the signal is applied only when the subject is in a specific state e.g. only when the subject is awake, only when the subject is asleep, prior to and/or after the ingestion of food, prior to and/or after the subject undertakes exercise, etc.

The various embodiments for timing for modulation of neural activity in the nerve can all be achieved using controller 101 in a system of the invention.

Other Components of the System Including the Implantable Device

In addition to the aforementioned at least one electrode (e.g. electrode 109) and microprocessor 113, the system 116 may comprise one or more of the following components: implantable transceiver 110; physiological sensor 111; power source 112; memory 114 (otherwise referred to as a non-transitory computer-readable storage device); and physiological data processing module 115. Additionally or alternatively, the physiological sensor 111; memory 114; and physiological data processing module 115 may be part of a sub-system external to the system. Optionally, the external sub-system may be capable of communicating with the system, for example wirelessly via the implantable transceiver 110.

In some embodiments, one or more of the following components may be contained in the implantable device 106: power source 112; memory 114; and a physiological data processing module 115.

The power source 112 may comprise a current source and/or a voltage source for providing the power for the signal delivered to the nerve by the at least one electrode (e.g. electrode 109). The power source 112 may also provide power for the other components of the implantable device 106 and/or system 116, such as the microprocessor 113, memory 114, and implantable transceiver 110. The power source 112 may comprise a battery, the battery may be rechargeable.

It will be appreciated that the availability of power is limited in implantable devices, and the invention has been devised with this constraint in mind. The implantable device 106 and/or system 116 may be powered by inductive powering or a rechargeable power source.

Memory 114 may store power data and data pertaining to the one or more physiological parameters from internal system 116. For instance, memory 114 may store data pertaining to one or more signals indicative of the one or more physiological parameters detected by physiological sensor 111, and/or the one or more corresponding physiological parameters determined via physiological data processing module 115. In addition or alternatively, memory 114 may store power data and data pertaining to the one or more physiological parameters from external system 118 via the implantable transceiver 110. To this end, the implantable transceiver 110 may form part of a communication subsystem of the wider system 100, as is further discussed below.

Physiological data processing module 115 is configured to process one or more signals indicative of one or more physiological parameters detected by the physiological sensor 111, to determine one or more corresponding physiological parameters. Physiological data processing module 115 may be configured for reducing the size of the data pertaining to the one or more physiological parameters for storing in memory 114 and/or for transmitting to the external system via implantable transceiver 110. Implantable transceiver 110 may comprise one or more antenna(e). The implantable transceiver 100 may use any suitable signaling process such as RF, wireless, infrared and so on, for transmitting signals outside of the body, for instance to wider system 100 of which the system 116 is one part.

Alternatively or additionally, physiological data processing module 115 may be configured to process the signals indicative of the one or more physiological parameters and/or process the determined one or more physiological parameters to determine the evolution of the disease in the subject. In such case, the system 116, in particular the implantable device 106, will include a capability of calibrating and tuning the signal parameters based on the one or more physiological parameters of the subject and the determined evolution of the disease in the subject.

The physiological data processing module 115 and the at least one physiological sensor 111 may form a physiological sensor subsystem, also known herein as a detector, either as part of the system 116, part of the implantable device 106, or external to the system.

In some embodiments, the device of the invention further comprises means to detect one or more physiological parameters in a subject. In some embodiments, the controller is coupled to said means to detect, and causes said one or more electrodes to apply said signal when the physiological parameter is detected to be meeting or exceeding a predefined threshold value. In some embodiments, the one or more detected physiological parameters comprise one or more of mean arterial blood pressure, heart rate, blood flow such as aortic blood flow and/or cortical blood flow, vascular resistance such as aortic vascular resistance and/or cortical vascular resistance, air flow, airway resistance, airway pressure such as upper airway pressure and/or lower airway pressure, diaphragmatic EMG and respiration rate

Physiological sensor 111 comprises one or more sensors, each configured to detect a signal indicative of one or more physiological parameters described above. For example, the physiological sensor 110 is configured for: detecting mean arterial blood pressure, heart rate, blood flow such as aortic blood flow and/or cortical blood flow, vascular resistance such as aortic vascular resistance and/or cortical vascular resistance, air flow, airway resistance, airway pressure such as upper airway pressure and/or lower airway pressure, diaphragmatic EMG and respiration rate.

The physiological parameters determined by the physiological data processing module 115 may be used to trigger the microprocessor 113 to deliver a signal of the kinds described above to the nerve using the at least one electrode (e.g. electrode 109). Upon receipt of the signal indicative of a physiological parameter received from physiological sensor 111, the physiological data processor 115 may determine the physiological parameter of the subject, and the evolution of the disease, by calculating in accordance with techniques known in the art. For instance, if a signal indicative of stimulation-mediated decreases in blood pressure or heart rate is detected, the processor may trigger delivery of a signal which lowers blood pressure.

The memory 114 may store physiological data pertaining to normal levels of the one or more physiological parameters. The data may be specific to the subject into which the system 116 is implanted, and gleaned from various tests known in the art. Upon receipt of the signal indicative of a physiological parameter received from physiological sensor 111, or else periodically or upon demand from physiological sensor 111, the physiological data processor 115 may compare the physiological parameter determined from the signal received from physiological sensor 111 with the data pertaining to a normal level of the physiological parameter stored in the memory 114, and determine whether the received signals are indicative of insufficient or excessive of a particular physiological parameter, and thus indicative of the evolution of the disease in the subject.

The system 116 and/or implantable device 106 may be configured such that if and when an insufficient or excessive level of a physiological parameter is determined by physiological data processor 115, the physiological data processor 115 triggers delivery of a signal to the nerve by the at least one electrode (e.g. electrode 109), in the manner described elsewhere herein. For instance, if physiological parameter indicative of worsening of any of the physiological parameters and/or of the disease is determined, the physiological data processor 115 may trigger delivery of a signal which lowers blood pressure, as described elsewhere herein. Particular physiological parameters relevant to the present invention are described above. When one or more signals indicative of one or more of these physiological parameters are received by the physiological data processor 115, a signal may be applied to the nerve via the at least one electrode (e.g. electrode 109).

In some embodiments, controller 101 may be configured to make adjustments to the operation of the system 116. For instance, it may transmit, via a communication subsystems (discussed further below), physiological parameter data pertaining to a normal blood pressure. The data may be specific to the patient into which the device is implanted. The controller 101 may also be configured to make adjustments to the operation of the power source 112, signal generator 117 and processing elements 113, 115 and/or electrode in order to tune the signal delivered to the nerve by the neural interface.

As an alternative to, or in addition to, the ability of the system 116 and/or implantable device 106 to respond to physiological parameters of the subject, the microprocessor 113 may be triggered upon receipt of a signal generated by an operator (e.g. a physician or the subject in which the system 116 is implanted). To that end, the system 116 may be part of a wider system 100 which comprises external system 118 and controller 101, as is further described below.

System Including Implantable Device

With reference to FIG. 1, the implantable device 106 of the invention may be part of a wider system 100 that includes a number of subsystems, for example the system 116 and the external system 118. The external system 118 may be used for powering and programming the system 116 and/or the implantable device 106 through human skin and underlying tissues.

The external subsystem 118 may comprise, in addition to controller 101, one or more of: a powering unit 102, for wirelessly recharging the battery of power source 112 used to power the implantable device 106; and, a programming unit 103 configured to communicate with the implantable transceiver 110. The programming unit 103 and the implantable transceiver 110 may form a communication subsystem. In some embodiments, powering unit 102 is housed together with programing unit 103. In other embodiments, they can be housed in separate devices.

The external subsystem 118 may also comprise one or more of: power transmission antenna 104; and data transmission antenna 105. Power transmission antenna 104 may be configured for transmitting an electromagnetic field at a low frequency (e.g., from 30 kHz to 10 MHz). Data transmission antenna 105 may be configured to transmit data for programming or reprogramming the implantable device 106, and may be used in addition to the power transmission antenna 104 for transmitting an electromagnetic field at a high frequency (e.g., from 1 MHz to 10 GHz). The temperature in the skin will not increase by more than 2 degrees Celsius above the surrounding tissue during the operation of the power transmission antenna 104. The at least one antennae of the implantable transceiver 110 may be configured to receive power from the external electromagnetic field generated by power transmission antenna 104, which may be used to charge the rechargeable battery of power source 112.

The power transmission antenna 104, data transmission antenna 105, and the at least one antennae of implantable transceiver 110 have certain characteristics such a resonant frequency and a quality factor (Q). One implementation of the antenna(e) is a coil of wire with or without a ferrite core forming an inductor with a defined inductance. This inductor may be coupled with a resonating capacitor and a resistive loss to form the resonant circuit. The frequency is set to match that of the electromagnetic field generated by the power transmission antenna 105. A second antenna of the at least one antennae of implantable transceiver 110 can be used in system 116 for data reception and transmission from/to the external system 118. If more than one antenna is used in the system 116, these antennae are rotated 30 degrees from one another to achieve a better degree of power transfer efficiency during slight misalignment with the with power transmission antenna 104.

External system 118 may comprise one or more external body-worn physiological sensors 121 (not shown) to detect signals indicative of one or more physiological parameters. The signals may be transmitted to the system 116 via the at least one antennae of implantable transceiver 110. Alternatively or additionally, the signals may be transmitted to the external system 116 and then to the system 116 via the at least one antennae of implantable transceiver 110. As with signals indicative of one or more physiological parameters detected by the implanted physiological sensor 111, the signals indicative of one or more physiological parameters detected by the external sensor 121 may be processed by the physiological data processing module 115 to determine the one or more physiological parameters and/or stored in memory 114 to operate the system 116 in a closed-loop fashion. The physiological parameters of the subject determined via signals received from the external sensor 121 may be used in addition to alternatively to the physiological parameters determined via signals received from the implanted physiological sensor 111.

For example, in a particular embodiment a detector external to the implantable device may include a non-invasive blood flow monitor, such as an ultrasonic flowmeter and/or a non-invasive blood pressure monitor, and determining changes in physiological parameters, in particular the physiological parameters described above. As explained above, in response to the determination of one or more of these physiological parameters, the detector may trigger delivery of signal to the nerve by the at least one electrode (e.g. electrode 109), or may modify the parameters of the signal being delivered or a signal to be delivered to the nerve by the at least one electrode in the future.

The wider system 100 may include a safety protection feature that discontinues the electrical stimulation of the nerve in the following exemplary events: abnormal operation of the system 116 (e.g. overvoltage); abnormal readout from an implanted physiological sensor 111 (e.g. temperature increase of more than 2 degrees Celsius or excessively high or low electrical impedance at the electrode-tissue interface); abnormal readout from an external body-worn physiological sensor 121 (not shown); or abnormal response to stimulation detected by an operator (e.g. a physician or the subject). The safety precaution feature may be implemented via controller 101 and communicated to the system 116, or internally within the system 116.

The external system 118 may comprise an actuator 120 (not shown) which, upon being pressed by an operator (e.g. a physician or the subject), will deliver a signal, via controller 101 and the respective communication subsystem, to trigger the microprocessor 113 of the system 116 to deliver a signal to the nerve by the at least one electrode (e.g. electrode 109).

Wider system 100 of the invention, including the external system 118, but in particular system 116, is preferably made from, or coated with, a biostable and biocompatible material. This means that the system is both protected from damage due to exposure to the body's tissues and also minimizes the risk that the system elicits an unfavorable reaction by the host (which could ultimately lead to rejection). The material used to make or coat the system should ideally resist the formation of biofilms. Suitable materials include, but are not limited to, poly(3,4-ethylenedioxythiophene):p-toluenesulfonate (PEDOT:PTS or PEDT), poly(p-xylylene) polymers (known as Parylenes) and polytetrafluoroethylene.

The implantable device 116 of the invention will generally weigh less than 50 g.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless otherwise indicated each embodiment as described herein may be combined with another embodiment as described herein.

### BRIEF DESCRIPTION OF THE DRAWINGS

Embodiments of the invention will be described, by way of example, with reference to the following drawings, in which:

FIG. 1 is a block diagram illustrating elements of a system for performing electrical modulation in the nerve according to the present invention.

FIG. 2 shows the effect of left renal nerve simulation on mean arterial pressure.

FIG. 3 shows the effect of left renal nerve stimulation on heart rate.

FIG. 4 shows the effect of left renal nerve stimulation on aortic (hindquarter) blood flow (ABF).

FIG. 5 shows the effect of left renal nerve stimulation on aortic (hindquarter) vascular resistance (AVR).

FIG. 6 shows the effect of left renal nerve stimulation on left renal cortical blood flow (CBF).

FIG. 7 shows the effect of left renal nerve stimulation on left renal cortical vascular resistance (CVR).

FIG. 8 shows the effect of left renal nerve stimulation on airway resistance (AR).

FIG. 9 shows the effect of left renal nerve stimulation on air flow (AF).

FIG. 10 shows the effect of left renal nerve stimulation on lower airway pressure (LAP).

FIG. 11 shows the effect of left renal nerve stimulation on upper airway pressure (UAP).

FIG. 12 shows the effect of left renal nerve stimulation on diaphragmatic EMG.

FIG. 13 shows the effect of left renal nerve stimulation on respiration rate (RR),

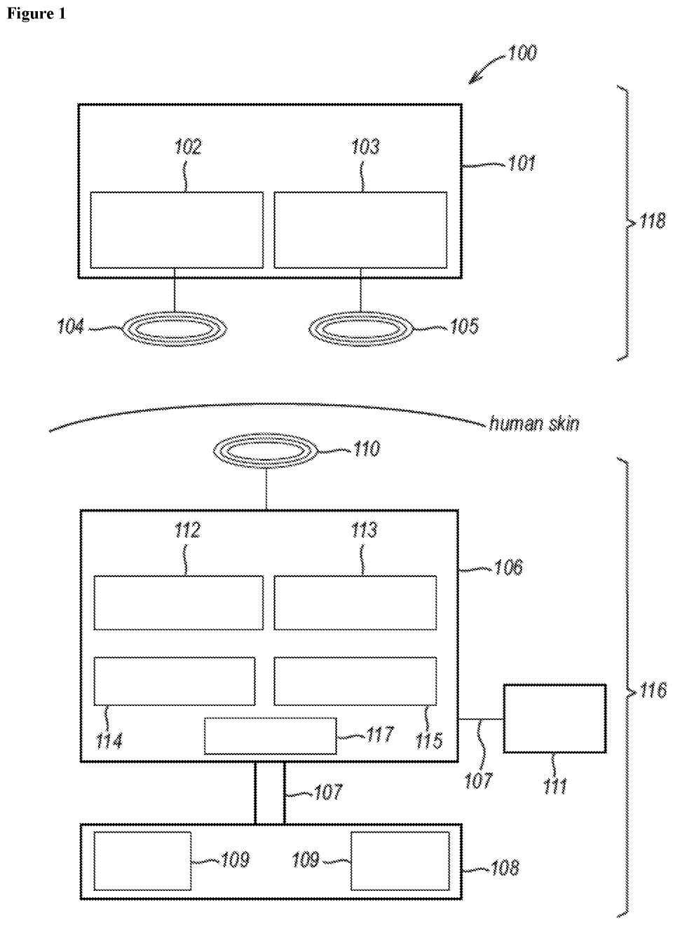

### MODES FOR CARRYING OUT THE INVENTION

Study 1—Assessing the Effect of Renal Nerve Stimulation in Spontaneously Hypertensive Rats

The inventors set out to test the effect on hypertension of stimulating neural activity in a renal nerve of a spontaneously hypertensive rat (SHR).

Method

Male spontaneously hypertensive rats (25-28 weeks of age) were anaesthetized with 50 mg/kg intraperitoneal injection of sodium pentobarbital and maintained with an intravenous infusion of 10 mg/kg/hr sodium pentobarbital into the right femoral vein. Mean arterial blood pressure and heart rate were measured via an intravenous cannula into the right carotid artery, left renal cortical blood flow (CBF) and cortical vascular resistance (CVR), hindquarter aortic blood flow (ABF) and vascular resistance (AVR), upper (UAP) and lower (LAP) airway pressure, airway flow (AF), airway resistance (AR), respiration rate (RR) and diaphragmatic EMG (dEMG). An algorithm that calculates the number of peaks per minute was applied on the integrated diaphragmatic EMG activity to calculate respiratory rate. A bipolar electrode was placed around the left renal nerve and stimulation delivered using a grass stimulator; 5 times (5-6 minutes apart) at 5 Hz, 0.5 ms, 0.5 mA for 30 s and responses were averaged.

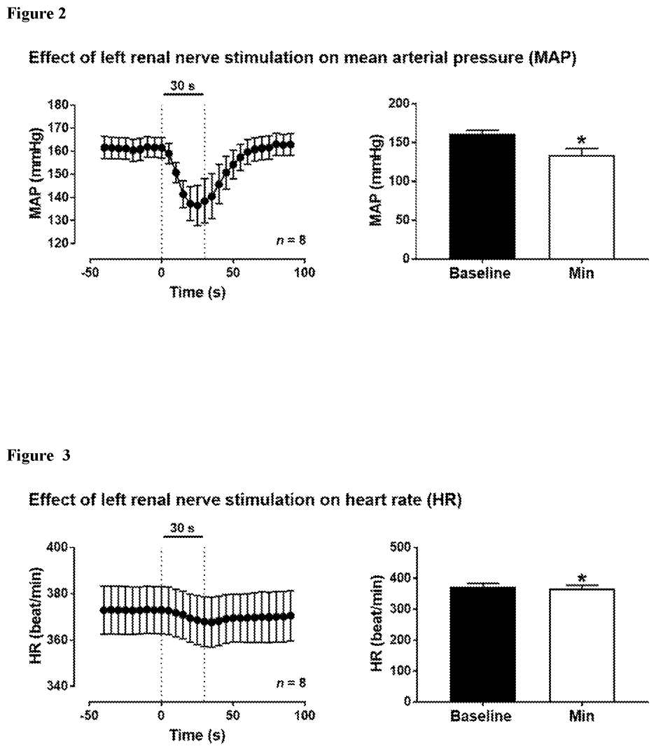

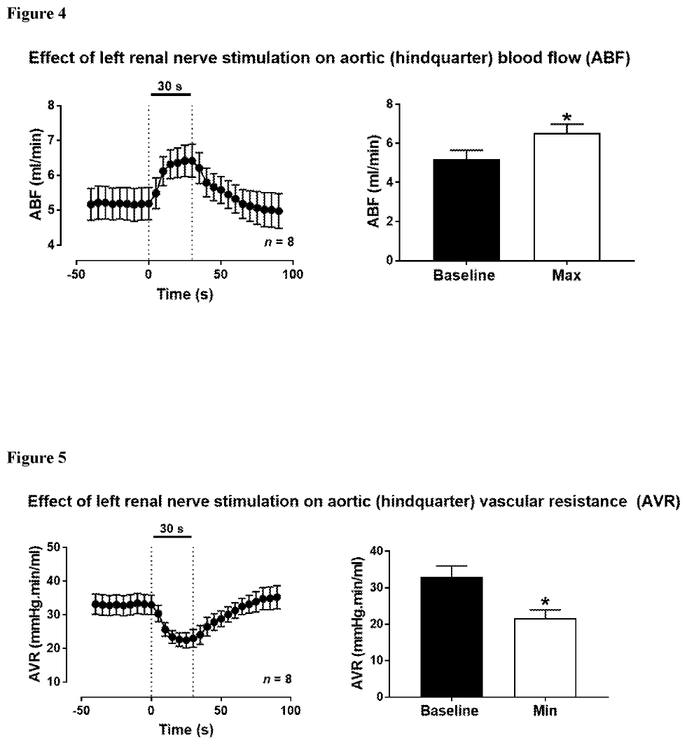

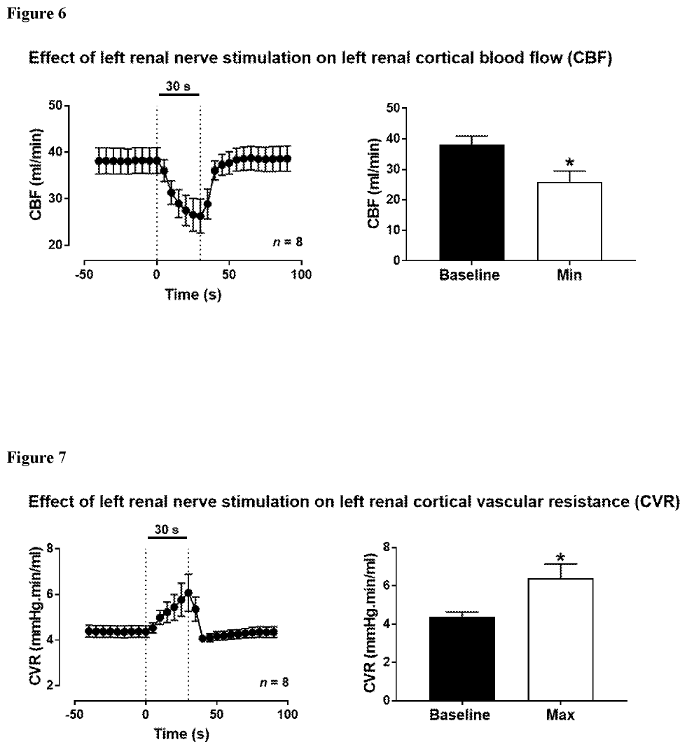

Cardiovascular responses to renal nerve stimulation exhibited reductions in mean arterial blood pressure (FIG. 2), heart rate (FIG. 3), aortic vascular resistance (FIG. 5) and cortical blood flow (FIG. 6). These responses were associated with increases in aortic blood flow (FIG. 4) and cortical vascular resistance (FIG. 7).

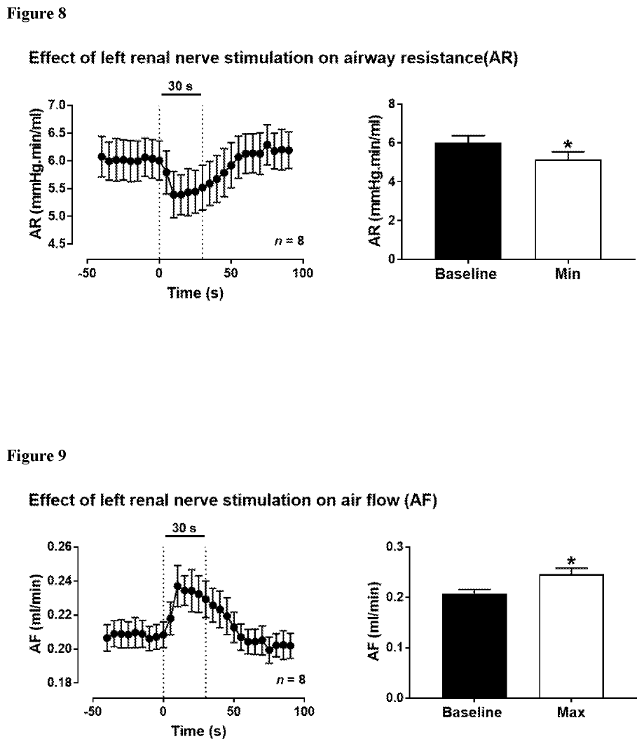

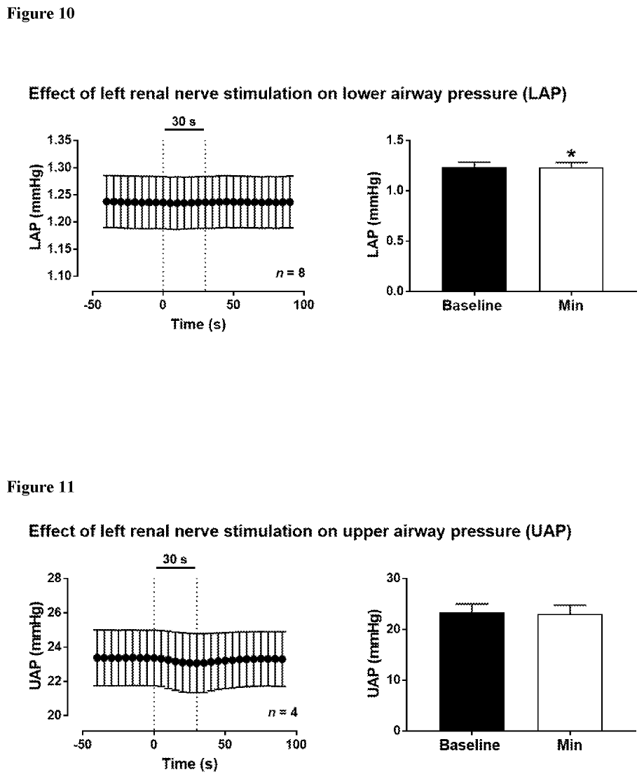

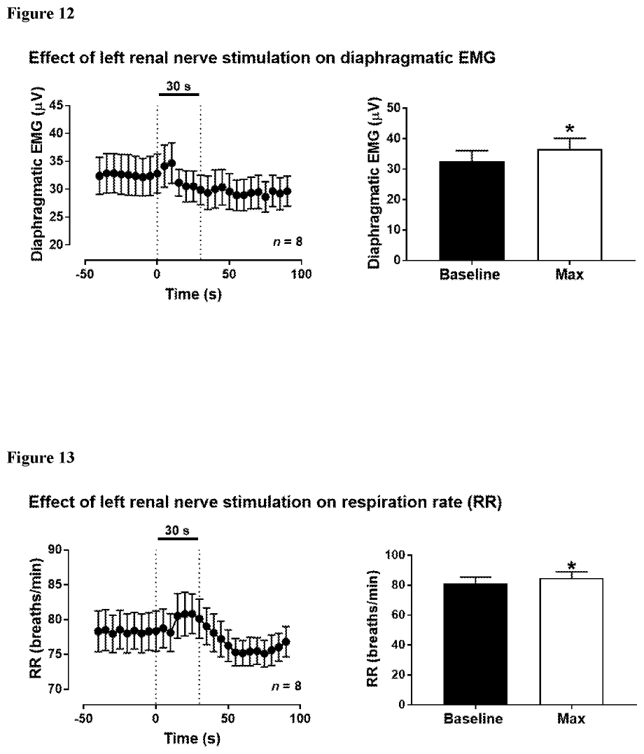

Respiratory responses to renal nerve stimulation exhibited increases in air flow (FIG. 9), respiration rate (FIG. 13) and diaphragmatic EMG (FIG. 12). These were accompanied by a decrease in airway resistance (FIG. 8). Lower airway pressure and upper airway pressure remained unaltered (FIGS. 10 and 11)

Discussion

The results show that, under hypertensive conditions, the role of renal nerves is not restricted to modulation of cardiovascular function but also extends to central neuroregulation of respiration and cardiorespiratory function.

Therefore, stimulation of a renal nerve may be a therapeutic strategy to alleviate hypertension, or symptoms thereof.

### REFERENCES

1 De Jong et al (2016) Hypertension 67:1211-1217
2 Gao et al (2019) Hypertension Research doi.org/10.1038/s41440-019-0237-3
3 Katholi et al (2009) Progress in cardiovascular disease 52:243-248
4 Esler et al (2010) Lancet 376: 1903-1909
5 Krum et al (2009) Lancet 373: 1275-81
6 Bhatt et al (2014) New England Journal of Medicine 270: 1390-1401
7 Persu et al (2015) Curr Opin Pharmacol 21:48-52
8 Shun-Shin et al (2014) BMJ 348:g1937
[9] Kramer et al., Optogenetic pharmacology for control of native neuronal signaling proteins, 2013; 16(7):816-23.

1-63. (canceled)

64. A device for stimulating the neural activity of a renal nerve, plexus or neurovascular bundle of a subject for treating hypertension and/or elevated blood pressure, and/or for treating a cardiorespiratory or cardiovascular disorder in a hypertensive subject, and/or for treating a cardiorespiratory or cardiovascular disorder in a subject with elevated blood pressure, the device comprising:
one or more electrodes in signaling contact with the renal nerve of the subject; and
a controller coupled to the one or more electrodes, the controller configured to control operation of the one or more electrodes to apply a signal in a periodic cycle such that the signal stimulates the neural activity of the renal nerve to produce a physiological response in the subject,
wherein the physiological response comprises a decrease in mean arterial blood pressure.

65. A device according to claim 64, wherein the signal comprises an alternating current (AC) waveform of from 0.2 Hz to 20 Hz frequency.

66. A device according to claim 65, wherein the signal comprises a pulse width of 2 ms or less.

67. A device according to claim 66, wherein the signal comprises a pulse width of from 0.4 ms to 2 ms.

68. A device according to claim 65, wherein the signal comprises a current of 1.0 mA to 30 mA.

69. A device according to claim 65, wherein the AC waveform is a sinusoidal waveform.

70. A device according to claim 64, wherein the periodic cycle comprises at least one signal period, wherein the signal is applied during the signal period.

71. A device according to claim 70, wherein the signal is applied continuously during the signal period.

72. A device according to claim 70, wherein the signal is applied in a burst pattern during the signal period, wherein the burst pattern comprises at least one burst in which the signal is on, and at least one rest period in which the signal is off, wherein the rest period begins at the end of a first burst, and ends at the beginning of a subsequent burst.

73. A device according to claim 64, wherein the device further comprises means to detect one or more physiological parameters in the subject, wherein the controller is coupled to said means to detect, and causes said one or more electrodes to apply said signal when the physiological parameter is detected to be meeting or exceeding a predefined threshold value, and further wherein the one or more detected physiological parameters comprise one or more of: mean arterial blood pressure, respiration rate, vascular resistance, hindquarter aortic blood flow, cortical blood flow, diaphragmatic EMG, or air flow.

74. A method of treating hypertension and/or elevated blood pressure in a subject, and/or of treating a cardiorespiratory or cardiovascular disorder in a hypertensive subject, and/or of treating a cardiorespiratory or cardiovascular disorder in a subject with elevated blood pressure, comprising:
i. implanting in the subject a device according to claim 64;
ii. positioning the one or more electrodes of the device in signaling contact with the renal nerve, plexus or neurovascular bundle of the subject; and
iii. activating the apparatus to stimulate neural activity of the renal nerve, plexus or neurovascular bundle in the subject.

75. A method according to claim 74, wherein the signal comprises an alternating current (AC) waveform of from 0.2 to 20 Hz frequency.

76. A method according to claim 75, wherein the signal comprises a pulse width of 2 ms or less.

77. A method according to claim 76, wherein the signal comprises a pulse width of from 0.4 ms to 2 ms.

78. A method according to claim 75, wherein the signal comprises a current of from 1.0 to 30 mA.

79. A method according to claim 75, wherein the AC waveform is a sinusoidal waveform.

80. A method according to claim 74, wherein the signal is applied in a periodic cycle comprising at least one signal period, wherein the signal is applied during the signal period.

81. A method according to claim 80, wherein the signal is applied continuously during the signal period.

82. A method according to claim 80, wherein the signal is applied in a burst pattern during the signal period, wherein the burst pattern comprises at least one burst in which the signal is on, and at least one rest period in which the signal is off, wherein the rest period begins at the end of a first burst, and ends at the beginning of a subsequent burst.

83. A method according to claim 74, further comprising the step of detecting one or more physiological parameters of the subject, wherein the signal is applied only when the detected physiological parameter meets or exceeds a predefined threshold value, wherein the device further comprises one or more detectors configured to detect the one or more physiological parameters, and further wherein the one or more detected physiological parameters comprises at least one of: mean arterial blood pressure, respiration rate, diaphragmatic EMG, hindquarter aortic blood flow, renal cortical blood flow, systemic vascular resistance, cortical vascular resistance, or air flow.
